(19) United States
(12) Patent Application Publication
(10) Pub. No.: US 2024/0329010 A1
(43) Pub. Date: Oct. 3, 2024

(54) SCANNING ASSEMBLY OF ULTRASONIC IMAGING DEVICE AND ULTRASONIC IMAGING DEVICE

(71) Applicant: GE Precision Healthcare LLC, Waukesha, WI (US)

(72) Inventors: Zuhua Wang, Wuxi (CN); Bo Dan, Wuxi (CN); Xin Wang, Wuxi (CN); Chunyan Qi, Wuxi (CN); Hongyu Zhao, Wuxi (CN)

(21) Appl. No.: 18/622,444

(22) Filed: Mar. 29, 2024

(30) Foreign Application Priority Data
Mar. 31, 2023 (CN) 202310347369.9

(51) Int. Cl. G01N 29/265; G01N 29/06; G01N 29/22

(52) U.S. Cl. CPC G01N 29/265; G01N 29/0654; G01N 29/225

(57) ABSTRACT

A scanning assembly of an ultrasonic imaging device including: a scanning unit configured to send an ultrasonic signal to a subject to be scanned and/or receives an ultrasonic signal reflected by the subject to be scanned; and a transparent screen disposed on the scanning unit, the transparent screen being configured to display information while the subject is being scanned and/or provide for observation of the scanning unit through the transparent screen.

### CROSS-REFERENCE TO RELATED APPLICATION

The present application claims priority to Chinese Patent Application No. 202310347369.9, filed on Mar. 31, 2023. The entire contents of the above-listed application are incorporated by reference herein in their entirety.

### TECHNICAL FIELD

The embodiments of the present application relate to the technical field of ultrasonic imaging, and in particular relate to a scanning assembly of an ultrasonic imaging device and the ultrasonic imaging device.

### BACKGROUND

An ultrasonic imaging device uses a scanning assembly to emit an ultrasonic signal and/or receive an ultrasonic signal so as to perform imaging on a subject to be scanned. When a user performs a scan by using the scanning assembly of the ultrasonic imaging device, it is generally necessary to know scan related information in order to operate the scanning assembly properly. Current ultrasonic imaging devices are provided with a display, and the user can observe content displayed on the display to know scan-related information. However, in an ultrasonic imaging device, the display and the scanning assembly are two independent components disposed separately, and the two are separated by a distance. The user needs to move the line of sight away from the scanning assembly in order to observe the content displayed on the display, and cannot simultaneously observe the display and the scanning assembly, thus resulting in reduced convenience of operation.

It should be noted that the above introduction of the background is only for the convenience of clearly and completely describing the technical solutions of the present application, and for the convenience of understanding for those skilled in the art.

### SUMMARY

Embodiments of the present application provide a scanning assembly of an ultrasonic imaging device and the ultrasonic imaging device.

According to an aspect of the embodiments of the present application, provided is a scanning assembly of an ultrasonic imaging device, comprising: a scanning unit that sends an ultrasonic signal to a subject to be scanned and/or receives an ultrasonic signal reflected by the subject to be scanned; and a transparent screen disposed on the scanning unit and configured to perform display while allowing the subject to be scanned and/or the scanning unit to be observed through the transparent screen.

According to an aspect of the embodiments of the present application, provided is an ultrasonic imaging device, comprising the scanning assembly described above.

With reference to the following description and drawings, specific implementations of the embodiments of the present application are disclosed in detail, and the means by which the principles of the embodiments of the present application can be employed are illustrated. It should be understood that the implementations of the present application are therefore not limited in scope. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, and constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive skill. In the drawings:

FIG. 1 is a schematic diagram of the structure of a scanning assembly of an ultrasonic imaging device according to embodiments of the present application;

FIG. 2 is another schematic diagram of the structure of the scanning assembly of the ultrasonic imaging device according to embodiments of the present application;

FIG. 3 is a schematic diagram of the structure of a driver and an ultrasonic transducer according to embodiments of the present application;

FIG. 4 is a schematic diagram of a connecting unit according to embodiments of the present application;

FIG. 5 is a schematic diagram of positioning information according to embodiments of the present application;

FIG. 6 is a schematic diagram of regional information according to embodiments of the present application;

FIG. 7 is a schematic diagram of a movement path of an ultrasonic transducer according to embodiments of the present application;

FIG. 8 is another schematic diagram of the regional information according to embodiments of the present application;

FIG. 9 is a schematic diagram of an ultrasonic imaging device according to embodiments of the present application;

FIG. 10 is a schematic diagram of a control method according to embodiments of the present application;

FIG. 11 is a schematic diagram of a host according to embodiments of the present application; and

FIG. 12 is another schematic diagram of the host according to embodiments of the present application.

### DETAILED DESCRIPTION

The foregoing and other features of the embodiments of the present application will become apparent from the following description and with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have” etc., refer to the presence of described features, elements, components, or assemblies, but do not exclude the presence or addition of one or more other features, elements, components, or assemblies.

In the embodiments of the present application, the singular forms “a” and “the”, etc. include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

In some embodiments, an ultrasonic imaging device may be used to perform scanning and imaging on a body part of the human body or other living entities. However, the present application is not limited thereto, and the ultrasonic imaging device may also be used to perform scanning and imaging on a non-living entity. Moreover, the present application is also applicable to other imaging devices having a similar structure or function (for example, devices for imaging by applying visible light, X-rays, magnetic fields, or other physical signals to a subject to be scanned).

One of the benefits of the embodiments of the present application is that by disposing a transparent screen on the scanning unit and configuring the transparent screen to perform display while allowing the subject to be scanned and/or the scanning unit to be observed therethrough, required information can be displayed by means of the transparent screen, such that a user, when operating the scanning unit, can obtain the required information by means of the transparent screen without moving the line of sight away from the scanning unit, thus facilitating an operation of the user. Moreover, the transparent screen does not block the line of sight of the user, so that the user can conveniently observe the subject to be scanned and/or the scanning unit.

The following is a specific description of embodiments of the present application with reference to the drawings.

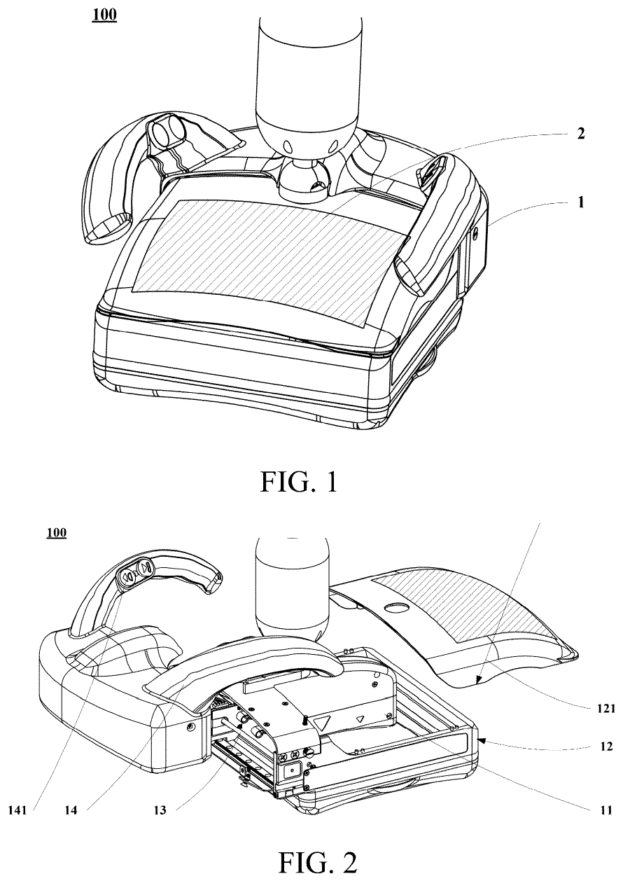

Provided in the embodiments of the present application is a scanning assembly of an ultrasonic imaging device. FIG. 1 is a schematic diagram of the structure of a scanning assembly of an ultrasonic imaging device according to embodiments of the present application. As shown in FIG. 1, the scanning assembly 100 includes a scanning unit 1 and a transparent screen 2. The scanning unit 1 sends an ultrasonic signal to a subject to be scanned and/or receives an ultrasonic signal reflected by the subject to be scanned. The transparent screen 2 is disposed on the scanning unit 1, and is configured to perform display while allowing the subject to be scanned and/or the scanning unit 1 to be observed through the transparent screen 2.

According to the above embodiment, by disposing the transparent screen 2 on the scanning unit 1 and configuring the transparent screen 2 to perform display while allowing the subject to be scanned and/or the scanning unit 1 to be observed through the transparent screen 2, required information can be displayed by means of the transparent screen 2, such that a user, when operating the scanning unit 1, can obtain the required information by means of the transparent screen 2 without moving the line of sight away from the scanning unit 1, thus facilitating an operation of the user. Moreover, the transparent screen 2 does not block the line of sight of the user, such that the user can conveniently observe the subject to be scanned and/or the scanning unit 1.

In some embodiments, the scanning unit 1 may apply the ultrasonic signal or the like to the subject to be scanned, and acquire signal strength distribution information fed back from the subject to be scanned. The acquired signal strength distribution information may be used by the scanning unit 1 or a host described below or other components to generate a scan image by using an image reconstruction technique or the like. The scan image may be a two-dimensional image or a three-dimensional image or a four-dimensional image, etc.

In some embodiments, the scanning unit 1 may be various structures capable of scanning the subject to be scanned. FIG. 2 is a schematic diagram of the structure of the scanning assembly of the ultrasonic imaging device according to embodiments of the present application. An exemplary description of the structure of the scanning unit 1 is given below with reference to FIG. 2. As shown in FIG. 2, the scanning unit I may include an ultrasonic transducer 11 and a housing 12. The ultrasonic transducer 11 may be used to send and/or receive an ultrasonic signal. The housing 12 may include a transparent material and cover the ultrasonic transducer 11 at least from the top, and the subject to be scanned and/or the ultrasonic transducer 11 can be observed through the housing 12. By configuring the housing 12 to be at least partially transparent and allow the subject to be scanned and/or the ultrasonic transducer 11 to be observed therethrough, it is easy to perform positioning of the scanning unit 1 and the subject to be scanned, and to learn the operating state of the ultrasonic transducer 11, etc.

In some embodiments, a preset scan range of the subject to be scanned is scanned by causing the ultrasonic transducer 11 to send and/or receive an ultrasonic signal within the preset scan range. The ultrasonic transducer 11 may cover the scan range in various ways. For example, the ultrasonic transducer 11 may be moved within the housing 12 in a preset mode of motion to cover the scan range. The preset mode of motion may be various modes of motion, for example, a reciprocating motion in a preset direction, a rotational motion around a preset center, or a combination of multiple modes of motion, etc. However, the present application is not limited thereto, and the ultrasonic transducer 11 may also not be moved. For example, the scan range may be covered by suitably setting an ultrasonic signal range.

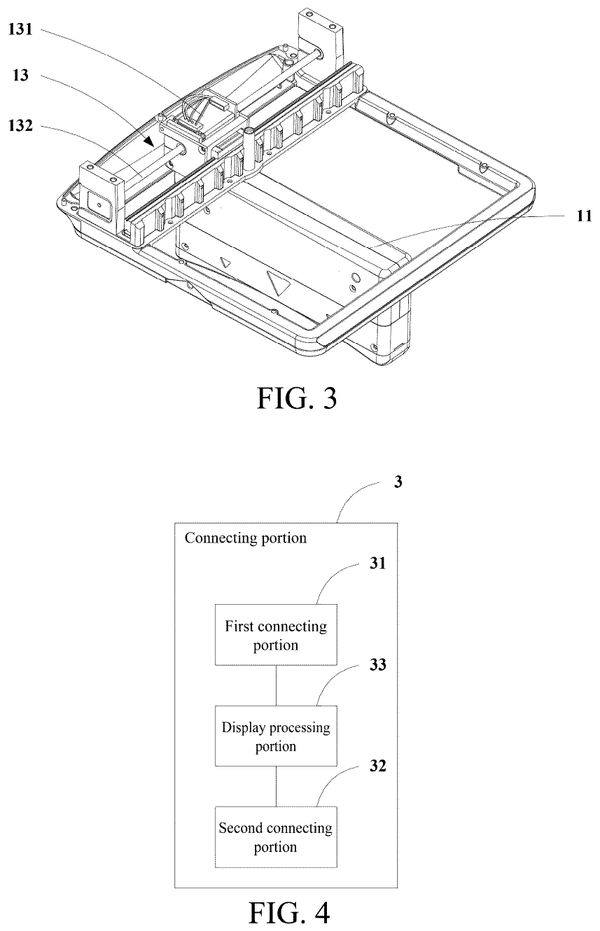

In some embodiments, as shown in FIG. 2, the scanning unit 1 may further include a driver 13 for driving the ultrasonic transducer 11 to move. The driver 13 may be various structures capable of driving the ultrasonic transducer 11 to move. FIG. 3 is a schematic diagram of the structure of the driver 13 and the ultrasonic transducer 11 according to embodiments of the present application. As shown in FIG. 3, the driver 13 may include a motor 131 and a lead screw 132. The motor 131 may be connected to the ultrasonic transducer 11, and is movably connected to the lead screw 132. Two ends of the lead screw 132 may be mounted on two sidewalls of the housing 12. The ultrasonic transducer 11 may be driven by the motor 131 to perform a reciprocating motion along the lead screw 132. The present application is not limited thereto, and the driver 13 may also be other structures, as long as the driver 13 can drive the ultrasonic transducer 11 to move and enable the scan range or range of motion (which may also be referred to as a range of movement) of the ultrasonic transducer 11 to cover the preset scan range.

In some embodiments, the ultrasonic transducer 11 may be located at an initial position in a non-scanning state. The initial position may be located at any position within the range of motion of the ultrasonic transducer 11. Taking the ultrasonic transducer 11 performing a reciprocating motion within the housing 12 as an example, the initial position may be located at a middle position of the range of motion, or at one end of the range of motion, etc.

In some embodiments, as shown in FIG. 2, the housing 12 may include a transparent material, i.e., at least one portion of the housing 12 is transparent. For example, an upper end cover 121 and/or sidewalls of the housing 12 may be transparent.

In some embodiments, as shown in FIG. 2, the scanning unit 1 may further include a handle 14, which may be disposed on the housing 12. By providing the handle 14, it is easy for the user to move the scanning unit 1, for example, to position the scanning unit 1 on the subject to be scanned or to move the scanning unit 1 away from the subject to be scanned.

In some embodiments, as shown in FIG. 2, the scanning unit 1 may further include a control unit 141 disposed on the handle 14. The control unit 141 may be operated to control the scanning unit 1, for example, to set scan parameters, to control scan progress, to set display parameters, etc. The control unit 141 may be in various forms, such as a button, a scroll wheel, or the like.

For case of understanding, an exemplary description of how to operate the scanning unit 1 is given. Before starting a scan, the user may position the scanning unit 1 at an appropriate position of the subject to be scanned. When the positioning of the two is maintained, the ultrasonic transducer 11 of the scanning unit 1 is moved within the housing 12 and sends and/or receives an ultrasonic signal.

In some embodiments, the scanning unit 1 may apply a preset pressure to the subject to be scanned during the scan. Such an arrangement can ensure that the ultrasonic signal is sent and/or received with less attenuation. In addition, the relative displacement of the scanning unit 1 and the subject to be scanned can be prevented, which facilitates maintaining the positioning of the scanning unit 1 and the subject to be scanned.

The above is merely an exemplary description of the structure and operation of the scanning unit 1. The present application is not limited thereto, and the scanning unit 1 may also be other structures, or include other components, or other ways may also be used to operate or use the scanning unit 1.

In some embodiments, the transparent screen 2 may be disposed on the housing 12 by means of a fixed connection. Alternatively, the transparent screen 2 may be at least one portion of the housing 12.

In some embodiments, the transparent screen 2 may be disposed on the top of the housing 12. By disposing the transparent screen 2 on the top of the housing 12, it is easy for the user to observe the subject to be scanned and/or the ultrasonic transducer 11 through the transparent screen 2 and the housing 12 while observing content displayed on the transparent screen 2, thereby helping the user to determine the positional relationship between the scanning unit 1 and the subject to be scanned, so as to facilitate the positioning of the scanning unit 1 and the subject to be scanned, and helping the user to understand the operating state of the ultrasonic transducer 11, so as to learn the scan progress, the scanning state, etc., which helps to increase the confidence of the user in the present scan, or enables the user to promptly identify a problem in the scan and intervene.

When the transparent screen 2 is disposed on the top of the housing 12, the transparent screen 2 may be disposed on an upper surface of the upper end cover 121 of the housing 12. Alternatively, the transparent screen 2 may be disposed on a lower surface of the upper end cover 121 of the housing 12, that is, the transparent screen 2 may be disposed within a receiving space of the housing 12. Alternatively, the transparent screen 2 may be disposed in the upper end cover 121 of the housing 12, that is, the transparent screen 2 may be a portion of the upper end cover 121. The present application is not limited thereto, and the transparent screen 2 may also be disposed elsewhere in the housing 12, for example, at the sidewalls of the housing 12, etc.

In some embodiments, the transparent screen 2 may be various transparent screens having a display function, for example, an LCD (Liquid Crystal Display) screen, an LED (light-emitting diode) screen, or the like. The transparent screen 2 may be a narrow-bezel screen, and the background color thereof may be close to the color of the housing 12, such that the transparent screen 2 does not interfere with the user's observation of the subject to be scanned and/or the scanning unit 1. The present application is not limited thereto, and the transparent screen 2 may also be other forms of screens.

In some embodiments, the scanning assembly 100 may further include a connecting unit for electrically connecting the transparent screen 2 to the scanning unit 1 and/or a host of the ultrasonic imaging device. FIG. 4 is a schematic diagram of the connecting unit according to embodiments of the present application. As shown in FIG. 4, the connecting unit 3 may include: a first connecting portion 31 and a second connecting portion 32. The first connecting portion 31 is electrically connected to the scanning unit 1 and/or the host of the ultrasonic imaging device, and the second connecting portion 32 electrically connects the first connecting portion 31 to the transparent screen 2. The arrangement of the connecting unit 3 can improve the degree of freedom in designing interfaces of the transparent screen 2 and the scanning unit 1 and/or the host. Even if the interface of the transparent screen 2 does not match the interface of the scanning unit 1 and/or the host, an electrical connection can be established therebetween by means of the connecting unit 3.

Taking the transparent screen 2 being an LCD screen as an example, the interface of the LCD screen is typically an FPC (flexible circuit board) cable, which has a large amount of signal data and cannot be directly connected to a control board of the scanning unit 1. By means of the connecting unit 3, the LCD screen and the scanning unit 1 can be conveniently connected.

The connecting unit 3 may be integrated with the LCD screen; or alternatively, may be disposed within the receiving space of the housing 12 or in the housing 12. The present application is not limited thereto, and the connecting unit 3 may also be disposed at other positions.

In some embodiments, when the connecting unit 3 electrically connects the transparent screen 2 to the scanning unit 1 and/or electrically connects the transparent screen 2 to the host, the scanning unit 1 and/or the host may power the transparent screen 2, and/or the scanning unit 1 and/or the host may communicate with the transparent screen 2.

In some embodiments, the first connecting portion 31 of the connecting unit 3 may be a hot-pluggable connecting portion which can be quickly connected to or disconnected from the transparent screen 2, thereby improving the convenience of using the transparent screen 2.

For example, the first connecting portion 31 may be a USB (Universal Serial Bus) interface, or the like. Taking the first connecting portion 31 being a USB TYPE-A interface as an example, a 5-pin male interface of the USB TYPE-A interface may include a power source, and a USB signal or UART (Universal Asynchronous Receiver-Transmitter) signal transceiving unit. The present application is not limited thereto, and the first connecting, 31 may also be other types of interfaces.

In some embodiments, as shown in FIG. 4, the connecting unit 3 may further include a display processor 33, which according to signals received from the scanning unit 1 and/or the host of the ultrasonic imaging device, may control the transparent screen 2 to perform display. The display processor 33 may be a microcontroller unit, or the like.

In some embodiments, the transparent screen 2 may include a touchscreen (not shown), which is capable of receiving information inputted by the user. Therefore, case of operation can be further improved.

In some embodiments, the transparent screen 2 may display various information, for example, may display auxiliary information related to the scan. The auxiliary information may include positioning information indicating a relative position of the scanning unit 1 with respect to the subject to be scanned, and/or regional information indicating a scan range of the scanning unit 1, etc. By displaying the above auxiliary information on the transparent screen 2, it is easy for the user to position the scanning unit 1 and the subject to be scanned, and/or for the user to know the scan range. For example, the user can conveniently observe the positioning information displayed on the transparent screen 2 and the subject to be scanned under the transparent screen 2 at the same time, and thus is able to conveniently position the scanning unit 1 and the subject to be scanned according to the positioning information. For another example, the user can conveniently observe the scan range displayed on the transparent screen 2 and the ultrasonic transducer 11 under the transparent screen 2 at the same time, so that it is easy for the user to confirm a current scan state.

In some embodiments, the positioning information may include an image corresponding to at least one portion of the subject to be scanned. The relative position of the scanning unit 1 with respect to the subject to be scanned may be a relative position of the scanning unit 1 with respect to the subject to be scanned when the image is aligned with the at least one portion of the subject to be scanned. By displaying the image corresponding to the at least one portion of a scanned subject on the transparent screen 2 disposed on the scanning unit 1, and aligning the image with the at least one portion of the scanned subject to position the scanning unit 1 and the subject to be scanned, it is helpful for the user to accurately and conveniently complete the positioning operation.

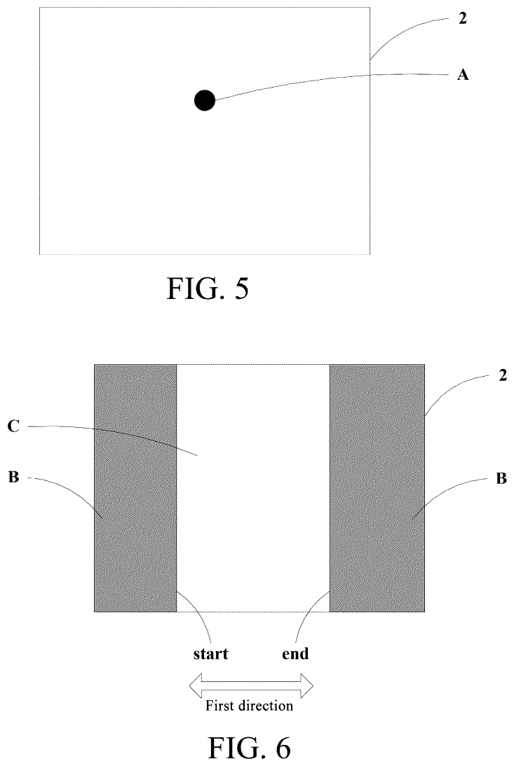

FIG. 5 is a schematic diagram of the positioning information according to embodiments of the present application. Taking the subject to be scanned being a breast as an example, as shown in FIG. 5, the image of the positioning information may be a dot A corresponding to a nipple. By adjusting the position of the scanning unit 1 to align the dot A with the nipple in an up-down direction, the scanning unit 1 is placed at a suitable position. The above merely uses the dot A to give an exemplary description of the image of the positioning information. It can be understood that the image of the positioning information may be other shapes, for example, a square, a triangle, a circle, a cross, or the like. Alternatively, the image of the positioning information may also be flexibly set according to different subjects to be scanned or usage habits of the user, etc.

The present application is not limited thereto, and the transparent screen 2 may also not display the positioning information. For example, the scanning unit 1 and the subject to be scanned may be positioned by using a marker of the scanning unit 1. For example, a hole may be provided in the housing 12, and since the hole may be observed through the transparent screen 2, the user may position the scanning unit 1 and the subject to be scanned by means of aligning the hole with at least one portion of the subject to be scanned, and the like.

In some embodiments, the regional information that indicates the scan range of the scanning unit 1 may include a starting position and an ending position, and between the starting position and the ending position is the scan range. The starting position, the scan range, and the ending position are arranged sequentially in a direction of motion of the ultrasonic transducer 11.

FIG. 6 is a schematic diagram of the regional information according to embodiments of the present application. As shown in FIG. 6, the ultrasonic transducer 11 may perform a reciprocating motion in a first direction. The regional information may include a starting position (start), an ending position (end), and a scan range C between the starting position (start) and the ending position (end), wherein the starting position (start), the scan range C, and the ending position (end) are arranged sequentially in the first direction.

The starting position (start) and the ending position (end) may be represented in various ways. For example, as shown in FIG. 5, shaded regions represent non-scan regions B, a non-shaded region represents a scan region C, and positions where the shaded regions abut the non-shaded region are the starting position (start) and the ending position (end). The above merely uses the shaded regions and the non-shaded region to give an exemplary description of the regional information. It can be understood that the regional information may be other shapes, for example, a marking line, a marking point, etc. FIG. 4 and FIG. 5 illustrate only examples in which the positioning information and the regional information are displayed separately. The present application is not limited thereto, and the positioning information and the regional information may be displayed simultaneously.

In some embodiments, the regional information of the scan range of the scanning unit 1 may be information corresponding to the size of the subject to be scanned, and the scan range indicated by the regional information may be less than or equal to a maximum scan range of the scanning unit. By making the regional information correspond to the actual size of the subject to be scanned, the scan range can be optimized, invalid scan regions can be removed, and the time required for scanning can be reduced. For example, if sizes of subjects to be scanned are not distinguished, then the maximum scan range needs to be applied for all the subjects to be scanned, and the time required to complete the scan is longer. By making the regional information correspond to the actual size of the subject to be scanned, a small scan range may be applied when the subject to be scanned is small, and a large scan range may be applied when the subject to be scanned is large, thereby facilitating an increase in scan efficiency.

In some embodiments, taking the ultrasonic transducer 11 performing a reciprocating motion within the housing 12 as an example, the maximum scan range of the ultrasonic transducer 11 is, for example, a range from a sidewall on one side to a sidewall on another side of the housing 12 in the direction of motion of the ultrasonic transducer 11, or the like.

In some embodiments, when the auxiliary information includes the positioning information and the regional information, the positioning information and the regional information may be associated with each other. For example, when one of the positioning information and the regional information changes, the other also changes accordingly therewith. Therefore, by adjusting one of the positioning information and the regional information, both the positioning information and the regional information can be adjusted to desired positions, thereby further improving the convenience of operation.

In some embodiments, the association information between the positioning information and the regional information may be preset. For example, the image of the positioning information may be always set at the center of the scan range represented by the regional information, and so on. When the position of the image of the positioning information is adjusted by the user, the scan range may be automatically adjusted. For example, when the position of the image is moved to the left by the user, the scan range may be automatically moved to the left as a whole by a corresponding distance. Alternatively, when the scan range is adjusted by the user, the position of the image of the positioning information corresponding to the scan range may be automatically adjusted. For example, when the scan range is expanded by the user, the center of the expanded scan range is automatically redetermined and the image of the positioning information is displayed at the newly determined center.

In some embodiments, the initial position of the ultrasonic transducer 11 of the scanning unit 1 may be included in the scan range. Therefore, the scan time can be further reduced.

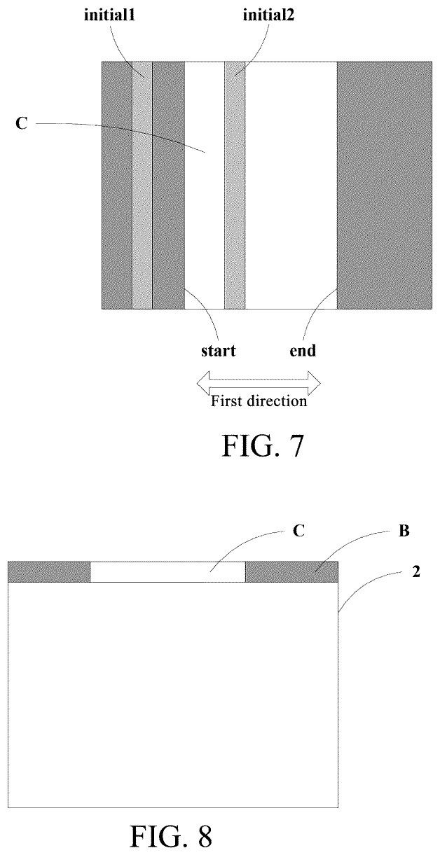

FIG. 7 is a schematic diagram of a movement path of the ultrasonic transducer 11 according to embodiments of the present application. As shown in FIG. 7, when the initial position of the ultrasonic transducer 11 is located at initial1, that is, the initial position of the ultrasonic transducer 11 is not located within the scan range C, to complete a scan of the scan range C, the ultrasonic transducer 11 needs to be moved according to the following path: initial1->start->end->start->initial1, that is, the distance the ultrasonic transducer 11 is moved is 2 times the distance between initial1 to end.

As shown in FIG. 7, when the initial position of the ultrasonic transducer 11 is located at initial2, that is, the initial position of the ultrasonic transducer 11 is located within the scan range C, to complete a scan of the scan range C, the ultrasonic transducer 11 needs to be moved according to the following path: initial2->start->end->initial2, that is, the distance the ultrasonic transducer 11 is moved is 2 times the distance between start to end. Therefore, by setting the initial position of the ultrasonic transducer 11 within the scan range C, the distance the ultrasonic transducer 11 is moved can be reduced, thereby enabling further reduction in the scan time.

In some embodiments, the range of movement of the ultrasonic transducer 11 during the scan may be determined according to the scan range. For example, during the scan, the range of movement of the ultrasonic transducer 11 may be included in the scan range, so that scanning of a non-scan range can be avoided, thereby enabling further reduction in the scan time. The present application is not limited thereto, and in the scanning process, the range of movement of the ultrasonic transducer 11 may also be slightly larger than the scan range. For example, the ultrasonic transducer 11 may start sending and/or receiving ultrasonic signals at a position further away from the ending position (end) than the starting position (start), and end sending and/or receiving ultrasonic signals at a position further away from the starting position (start) than the ending position (end). Thus, the reliability of the ultrasonic signals throughout the scan range from the starting position (start) to the ending position (end) and the integrity of the signals within the scan range can be ensured.

In some embodiments, the velocity of movement of the ultrasonic transducer 11 may vary within different movement intervals. For example, the ultrasonic transducer 11 may not send and/or receive ultrasonic signals in a process of moving from the initial position initial1 or initial2 to the starting position (start) of the scan range C, and/or in a process of the ultrasonic transducer 11 returning from the ending position (end) of the scan range C back to the initial position initial1 or initial2, and thus, in the process, the ultrasonic transducer 11 may be moved at a relatively higher velocity V1. The ultrasonic transducer 11 may send and/or receive ultrasonic signals in a process of moving from the starting position (start) to the ending position (end), and thus, in the process, the ultrasonic transducer 11 may be moved at a relatively lower velocity V2, i.e., V1>V2. Therefore, the time required to complete the scan can be further reduced while ensuring the reliability of the scan result. The present application is not limited thereto, and the velocity of movement of the ultrasonic transducer 11 may also be the same within different movement intervals.

In some embodiments, during the scan, the auxiliary information may further include at least one of: real-time ultrasonic images, scan state information, or parameter information during the scan. Therefore, the convenience of operation can be further improved. If the above auxiliary information is displayed by using a separate display screen, the user will move the line of sight to observe the same. However, the time allowed for the user to operate the scanning assembly 100 (for example, adjusting the parameters, etc.) during the scan is short. Compared to the separate display screen, by displaying the above auxiliary information on the transparent screen 2, the user can know the above auxiliary information without moving the line of sight away from the scanning assembly 100, so that it is easy for the user to promptly operate the scanning assembly 100 according to the auxiliary information. For example, the user may perform control using the control unit on the handle 14 of the scanning assembly 100 and/or the touchscreen in the transparent screen 2, etc.

In some embodiments, the scan state information may include the scan progress (scan start, scan pause, scan completion, re-scan, remaining scan time, expected scan duration, etc.), or the scan result (successful scan, failed scan, etc.), or other information. The parameter information during the scan may include the parameter information of the scanning unit 1 (contact pressure between the scanning unit 1 and the subject to be scanned, scan speed, ultrasonic signal parameters, etc.), the parameter information of the transparent screen 2 (display parameters, content/mode adjustment parameters, etc.), or other information. The present application is not limited thereto, and the scan state information, the parameter information during the scan or the auxiliary information may also include other content or may be set by the user.

In some embodiments, in the scanning process, the positioning information and/or the regional information may be displayed as zoomed out. Therefore, other auxiliary information can be displayed on the transparent screen 2. FIG. 8 is another schematic diagram of the regional information according to embodiments of the present application. An exemplary description of a display manner of the transparent screen 2 is given below with reference to FIG. 8. For example, before a scanning process starts, the transparent screen 2 may display the regional information representing the scan range in a larger range, and may also display the image of the positioning information for positioning the scanning unit 1 and the subject to be scanned. As shown in FIG. 8, after the scanning process starts, the transparent screen 2 may reduce the range of displaying the regional information and/or the positioning information, and display other auxiliary information in other regions of the transparent screen 2. For example, the image of the positioning information is not displayed, the regional information is displayed by only using a strip-shaped region at the top of the transparent screen 2, real-time ultrasonic images are displayed within other regions of the transparent screen 2, and so on. The present application is not limited thereto, and the positioning information and/or the regional information may also be otherwise displayed as zoomed out.

According to the above embodiment, by disposing the transparent screen 2 on the scanning unit 1 and configuring the transparent screen 2 to perform display while allowing the subject to be scanned and/or the scanning unit 1 to be observed through the transparent screen 2, required information can be displayed by means of the transparent screen 2, such that a user, when operating the scanning unit 1, can obtain the required information by means of the transparent screen 2 without moving the line of sight away from the scanning unit 1, which facilitates an operation of the user. Moreover, the transparent screen 2 does not block the line of sight of the user, so that the user can observe the subject to be scanned and/or the scanning unit 1 easily.

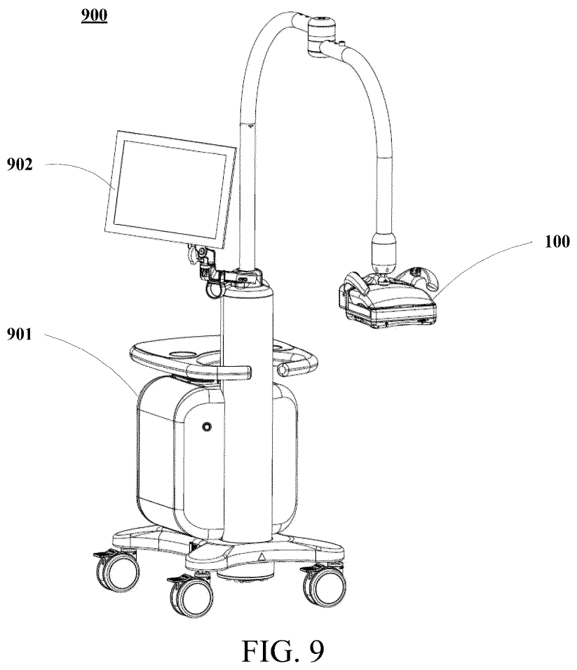

Further provided in the embodiments of the present application is an ultrasound imaging system. FIG. 9 is a schematic diagram of the ultrasonic imaging device according to embodiments of the present application. As shown in FIG. 9, the ultrasonic imaging device 900 may include the scanning assembly 100 as described above. Since the above scanning assembly 100 is provided in the ultrasonic imaging device 900, the required information can be displayed by means of the transparent screen of the scanning assembly 100, such that the user, when operating the scanning unit of the scanning assembly 100, can obtain the required information by means of the transparent screen without moving the line of sight away from the scanning unit, which facilitates the operation of the user. Moreover, the transparent screen does not block the line of sight of the user, so that the user can observe the subject to be scanned and/or the scanning unit easily, thereby improving the convenience of operation of the ultrasonic imaging device 900.

In some embodiments, as shown in FIG. 9, the ultrasonic imaging device 900 may further include a host 901. The host 901 may be electrically connected to the scanning assembly 100 to control the scanning unit 1 and/or the transparent screen 2 of the scanning assembly 100. For example, the host 901 may control the transparent screen 2 to display auxiliary information, and/or control the motion of the ultrasonic transducer 11 of the scanning unit 1.

Below, taking the host 901 controlling the transparent screen 2 as an example, an exemplary description of a control method for the host 901 is given.

The host 901 may generate scan-related auxiliary information so as to display the same on the transparent screen 2 of the scanning assembly 100. The auxiliary information may include positioning information indicating a relative position of the scanning unit 1 with respect to the subject to be scanned, and/or regional information indicating a scan range of the scanning unit 1.

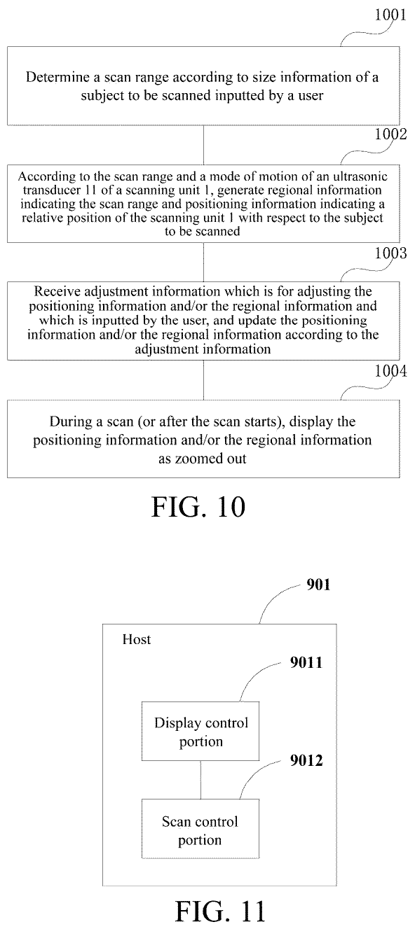

The host 901 may generate the positioning information and the regional information in the following manner. FIG. 10 is a schematic diagram of a control method according to embodiments of the present application. As shown in FIG. 10, the control method includes:

1001, determining a scan range according to size information of a subject to be scanned inputted by a user; and

1002, according to the scan range and a mode of motion of an ultrasonic transducer 11 of a scanning unit 1, generating regional information indicating the scan range and positioning information indicating a relative position of the scanning unit 1 with respect to the subject to be scanned.

Therefore, the transparent screen 2 may display the positioning information and the regional information at appropriate positions.

In some embodiments, in 1001, the size information of the subject to be scanned

inputted by the user is, for example, large, medium, small, or other information, and the host 901 may generate a corresponding scan range according to the size information. The correspondence between the size information and the scan range may be pre-stored information or calculated according to information inputted by the user.

In some embodiments, in 1002, the regional information indicating the scan range of the scanning unit 1 may include a starting position and an ending position, and the starting position, the scan range between the starting position and the ending position, and the ending position may be arranged sequentially in a direction of motion of the ultrasonic transducer 11. Therefore, the scan range can be clearly represented.

In some embodiments, in 1002, the starting position of the regional information may be set at a current position (for example, the initial position) of the ultrasonic transducer 11 or near the current position of the ultrasonic transducer 11, and thus, the distance the ultrasonic transducer 11 is moved can be reduced, thereby further reducing the time required for the scan.

In some embodiments, the positioning information indicating a relative position of the scanning unit 1 with respect to the subject to be scanned may include an image corresponding to at least one portion of the subject to be scanned, and the relative position may be a relative position of the scanning unit 1 with respect to the subject to be scanned when the image is aligned with the at least one portion of the subject to be scanned. In 1002, the position of the image of the positioning information may be determined according to the regional information and the positional relationship between the regional information and the at least one portion of the subject to be scanned. Alternatively, the position of the image of the positioning information may be determined first, and then the regional information is determined according to the position of the image of the positioning information. The positional relationship between the regional information and the at least one portion of the subject to be scanned may be pre-stored information or information obtained by calculation.

In some embodiments, the control method may further include:

1003, receiving adjustment information which is for adjusting the positioning information and/or the regional information and which is inputted by the user, and updating the positioning information and/or the regional information according to the adjustment information.

In some embodiments, the adjustment information may be information inputted by the user by operating the touchscreen of the transparent screen 2. For example, when the transparent screen 2 displays the positioning information and/or regional information, the user may adjust a display position of the positioning information and/or the regional information by means of dragging, clicking, or other operations so as to adjust the image of the positioning information to suitable position or adjust the regional information to a suitable range. The present application is not limited thereto, and the adjustment information may also be information inputted by the user by operating the control unit 141 of the handle 14 of the scanning unit 1 or other input devices of the ultrasonic imaging device 900 (for example, a mouse, a keyboard, a touchscreen of a display which will be described below of the ultrasonic imaging device 900, etc.).

In some embodiments, the positioning information and the regional information may be associated with each other. By associating the positioning information and the regional information with each other, when one of the two changes, the other also changes accordingly therewith. Therefore, by adjusting one of the positioning information and the regional information, both the positioning information and the regional information can be adjusted to desired positions, thereby further improving the convenience of operation.

In some embodiments, during the scan, the auxiliary information further includes at least one of: real-time ultrasonic images, scan state information, or parameter information during the scan. In order to enable proper display of the above auxiliary information, the control method may further include:

1004, during the scan (or after the scan starts), displaying the positioning information and/or the regional information as zoomed out.

It should be noted that FIG. 10 above merely schematically illustrates the embodiment of the present application, but the present application is not limited thereto. For example, the order of execution between operations may be suitably adjusted. In addition, some other operations may also be added or some of these operations may be omitted. In addition, the subject of the above operations may also be adjusted. Those skilled in the art can make appropriate variations according to the above content rather than being limited by the disclosure of FIG. 10 above.

Taking the host 901 controlling the ultrasonic transducer 11 of the scanning unit 1 as an example, an exemplary description of a control method for the host 901 is given below.

In some embodiments, the host 901 may control the range of movement of the ultrasonic transducer 11 of the scanning unit 1 according to the regional information. Taking the ultrasonic transducer 11 performing a reciprocating motion within the housing 12 as an example, the host may cause the ultrasonic transducer 11 to move from an initial position to one end 11 (for example, the starting position (start)) of the scan range indicated by the regional information displayed on the transparent screen 2 in the direction of movement of the ultrasonic transducer, cause the ultrasonic transducer 11 to move from the one end to another end (for example, the ending position (end)) of the scan range in the direction of movement, and cause the ultrasonic transducer 11 to return from the other end to the initial position. The present application is not limited thereto, and the host 901 may otherwise control the motion of the ultrasonic transducer 11.

In some embodiments, during the scan, the host 901 may control the range of movement of the ultrasonic transducer 11, so that the range of movement is included in the scan range.

FIG. 11 is a schematic diagram of the host 901 according to embodiments of the present application. As shown in FIG. 11, the host may include a display control portion 9011 and a scan control portion 9012. The display control portion 9011 and the scan control portion 9012 implement at least a function of controlling the transparent screen 2 to display the auxiliary information and a function of controlling the motion of the ultrasonic transducer 11 of the scanning unit 1, respectively.

In some embodiments, the display control portion 9011 may also receive raw image data (for example, an ultrasonic echo) from the scanning assembly 100 in addition to controlling the transparent screen 2. The display control portion 9011 may process the raw image data to form a displayable image, and/or send the raw image data to a remote processor (for example, via a network) to generate a displayable image. The displayable image may be displayed by the aforementioned transparent screen 2 and/or by a display described below.

In some embodiments, the scan control portion 9012 may also control other actions of the ultrasonic transducer 11 in addition to controlling the mode of motion of the ultrasonic transducer 11. For example, the scan control portion 9012 may activate or deactivate a transducer element of the ultrasonic transducer 11 to cause the ultrasonic transducer 11 to send or stop sending an ultrasonic signal, etc.

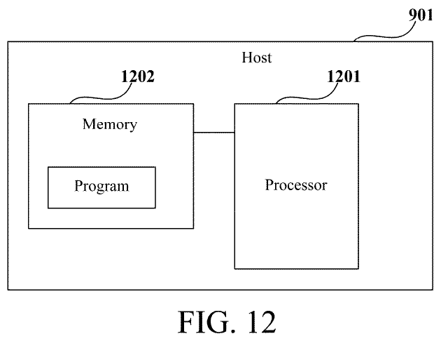

FIG. 12 is another schematic diagram of the host 901 according to embodiments of the present application. As shown in FIG. 12, the host 901 may include: one or more processors (for example, a central processing unit (CPU)) 1201, and one or more memories 1202 coupled to the processor 1201. The memory 1202 may store various types of information, etc. In addition, the memory may further store a control program of a device, and the program is executed under the control of the processor 1201 to control the scanning unit 1 and/or the transparent screen 2. For example, the functions of the above display control portion 9011 and/or the scan control portion 9012 are integrated into the program. The memory 1202 may include, for example, a ROM, a floppy disk, a hard disk, an optical disk, a magneto-optical disk, a CD-ROM, or a non-volatile memory card. It should be noted that the host 901 does not necessarily include all of the components shown in FIG. 12. In addition, the host 901 may further include components not shown in FIG. 12, for which reference may be made to the related technologies.

For the sake of simplicity, FIG. 12 only exemplarily illustrates the connection relationship or signal direction between various components or modules, but it should be clear to those skilled in the art that various related technologies such as bus connection can be used. The various components or modules can be implemented by means of a hardware facility such as a processor or a memory, etc. The embodiments of the present application are not limited thereto.

As shown in FIG. 9, in some embodiments, the ultrasonic imaging device may further include a display 902. The scanning assembly 100, the display 902, and the host 901 are independent components communicating with each other; however, in some embodiments, one or more of these components may be integrated (for example, the display 902 and the host 901 may be included in a single component).

In some embodiments, the display 902 may display information the same as, or different from, the content displayed by the transparent screen 2. For example, the display 902 may display more detailed information than the transparent screen 2, so that the user may use the display 902 to know the above information when necessary.

The display 902 may include a user interface configured to display images or other information to the user. Furthermore, the user interface may be configured to receive input from the user and send the input to the host 901. In some embodiments, the user interface may be a touchscreen of the display 110.

The above is merely an exemplary description of the ultrasonic imaging device 900. The present application is not limited thereto, and the ultrasonic imaging device 900 may also not be provided with the display 110, or the ultrasonic imaging device 900 may include other components.

The embodiments of the present application further provide a computer-readable program, wherein when the program is executed, the same causes a computer to perform, in the ultrasonic imaging device, the control method described in the foregoing embodiments.

The embodiments of the present application further provide a storage medium storing a computer-readable program, wherein the computer-readable program causes a computer to perform, in the ultrasonic imaging device, the control method described in the foregoing embodiments.

The above device and method of the present application can be implemented by hardware, or can be implemented by hardware in combination with software. The present application relates to the foregoing type of computer-readable program. When executed by a logic component, the program causes the logic component to implement the foregoing device or a constituent component, or causes the logic component to implement various methods or steps as described above. The present application further relates to a storage medium for storing the above program, such as a hard disk, a disk, an optical disk, a DVD, a flash memory, etc.

The method/device described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by curing the software modules using a field-programmable gate array (FPGA).

The software modules may be located in a RAM, a flash memory, a ROM, an EPROM, an EEPROM, a register, a hard disk, a removable disk, a CD-ROM, or any other form of storage medium known in the art. The storage medium may be coupled to a processor, so that the processor can read information from the storage medium and can write information into the storage medium. Alternatively, the storage medium may be a component of the processor. The processor and the storage medium may be located in an ASIC. The software module may be stored in a memory of a mobile terminal, and may also be stored in a memory card that can be inserted into the mobile terminal. For example, if a device (such as a mobile terminal) uses a large-capacity MEGA-SIM card or a large-capacity flash memory device, the software modules can be stored in the MEGA-SIM card or the large-capacity flash memory device.

One or more functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principles of the present application, and these variations and modifications also fall within the scope of the present application.

The above embodiments merely provide illustrative descriptions of the embodiments of the present application. However, the present application is not limited thereto, and appropriate variations may be made on the basis of the above embodiments. For example, each of the above embodiments may be used independently, or one or more of the above embodiments may be combined.

Embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

What is claimed is:

1. A scanning assembly of an ultrasonic imaging device, comprising:
a scanning unit configured to send an ultrasonic signal to a subject to be scanned and/or receives an ultrasonic signal reflected by the subject to be scanned; and
a transparent screen disposed on the scanning unit, the transparent screen being configured to display information while the subject is being scanned and/or provide for observation of the scanning unit through the transparent screen.

2. The scanning assembly according to claim 1, wherein the scanning unit comprises:
an ultrasonic transducer that sends and/or receives the ultrasonic signal; and
a housing comprising a transparent material, the housing covering the ultrasonic transducer at least from the top, and provides for observation of the ultrasonic transducer therethrough,
wherein the transparent screen is disposed on the top of the housing.

3. The scanning assembly according to claim 1, wherein
the transparent screen is configured to display auxiliary information related to a scan, the auxiliary information comprising positioning information indicating a relative position of the scanning unit with respect to the subject to be scanned, and/or regional information indicating a scan range of the scanning unit.

4. The scanning assembly according to claim 3, wherein
the positioning information comprises an image corresponding to at least one portion of the subject to be scanned, and the relative position is a relative position of the scanning unit with respect to the subject to be scanned which enables the image to be aligned with the at least one portion of the subject to be scanned.

5. The scanning assembly according to claim 3, wherein
the regional information is information corresponding to the size of the subject to be scanned, and the scan range indicated by the regional information is less than or equal to a maximum scan range of the scanning unit.

6. The scanning assembly according to claim 3, wherein
the auxiliary information comprises the positioning information and the regional information, and the positioning information and the regional information are associated with each other.

7. The scanning assembly according to claim 3, wherein
an initial position of an ultrasonic transducer of the scanning unit is included in the scan range, and/or
during the scan, a range of movement of the ultrasonic transducer is included in the scan range.

8. The scanning assembly according to claim 3, wherein
during the scan, the auxiliary information further comprises at least one of: a real-time ultrasonic image, scan state information, or parameter information during the scan.

9. The scanning assembly according to claim 8, wherein the positioning information and/or the regional information are displayed as zoomed out.

10. The scanning assembly according to claim 8, further comprising:
a handle and a control unit which is disposed on the handle and which can be operated to control the scanning unit.

11. The scanning assembly according to claim 1, further comprising:
a connecting unit comprising:
a first connecting portion electrically connected to the scanning unit and/or a host of the ultrasonic imaging device; and
a second connecting portion electrically connecting the first connecting portion to the transparent screen.

12. The scanning assembly according to claim 11, wherein
the first connecting portion is a hot-pluggable connecting portion.

13. The scanning assembly according to claim 11, wherein
responsive to the connecting unit electrically connecting the transparent screen to the scanning unit and/or the transparent screen to the host, i) the scanning unit and/or the host powers on the transparent screen, and/or ii) the scanning unit and/or the host communicates with the transparent screen.

14. The scanning assembly according to claim 1, wherein
the transparent screen comprises a touchscreen capable of receiving information inputted by a user.

15. An ultrasonic imaging device, comprising the scanning assembly according claim 1.

16. The device according to claim 15, wherein the ultrasonic imaging device further comprises:
a memory storing instruction; and
a processor configured to execute the instructions to:
generate scan-related auxiliary information to be displayed by a transparent screen; and
control the transparent screen to display the scan-related auxiliary information.

17. The device according to claim 16, wherein
the processor is further configured to execute the instructions to control a range of movement of an ultrasonic transducer of a scanning unit according to regional information.

18. The device according to claim 17, wherein
the processor is further configured to execute the instructions to cause the ultrasonic transducer to move from an initial position to one end of a scan range indicated by the regional information in a direction of movement of the ultrasonic transducer, cause the ultrasonic transducer to move from the one end to another end of the scan range in the direction of movement, and cause the ultrasonic transducer to return from the other end to the initial position.
